(19) United States
(12) Patent Application Publication
(10) Pub. No.: US 2002/0002331 A1
(43) Pub. Date: Jan. 3, 2002

(54) MR IMAGING WITH PARTIAL K-SPACE ACQUISITION USING SPIRAL SCANNING

(76) Inventors: HARVEY ELLIS CLINE, SCHENECTADY, NY (US); SARAH KATHRYN PATCH, SARATOGA SPRINGS, NY (US); CHRISTOPHER JUDSON HARDY, SCHENECTADY, NY (US); WILLIAM ALAN EDELSTEIN, SCHENECTADY, NY (US)

Correspondence Address: GENERAL ELECTRIC COMPANY, CRD PATENT DOCKET RM 4Q59, PO BOX 8 BLDG K-1-SALAMONE, SCHENECTADY, NY, 12301

(21) Appl. No.: 09/312,687

(22) Filed: May 14, 1999

(51) Int. Cl. A61B 5/055

(52) U.S. Cl. 600/410; 324/307; 324/309

(57) ABSTRACT

An MRI system acquires image data using a spiral pulse sequence in which k-space is sampled in a trajectory comprised of a spiral segment and a symmetric spiral tail segment. The spiral segments partially sample throughout the extent of k-space and the symmetric spiral tail segment samples only in a central region of k-space. The central region of k-space is fully sampled and a phase correction method is used to reconstruct an image from the under-sampled peripheral k-space data set.

### BACKGROUND OF THE INVENTION

This invention relates to nuclear magnetic resonance imaging methods and systems and, more particularly, to acquisition of images using spiral scanning methods.

When a substance such as human tissue is subjected to a uniform magnetic field (polarizing field B₀), the individual magnetic moments of the spins in the tissue attempt to align with this polarizing field along a longitudinal z axis, but precess about it in random order at their characteristic Larmor frequency. If the substance, or tissue, is subjected to a magnetic field (excitation field B₁) which is in the x-y plane and which is near the Larmor frequency, the net aligned moment M_{z} may be rotated, or “tipped”, into the x-y plane to produce a net transverse magnetic moment Mₜ. A nuclear magnetic resonance (NMR) signal is emitted by the excited spins after the excitation signal B₁ is terminated, and may be received and processed to form an image.

When utilizing NMR signals to produce images, magnetic field gradients (Gₓ G_{y} and G_{z}) are employed. Typically, the region to be imaged is scanned by a sequence of measurement cycles in which these gradients vary according to the particular localization method being used to sample a two or three dimensional region of k-space. The resulting set of received k-space signals is digitized and processed to reconstruct the image using one of many well known reconstruction techniques.

Most magnetic resonance (MR) scans used to produce medical images require many minutes to acquire the necessary k-space data. Reducing this scan time is an important objective, since a shortened scan increases patient throughput, improves patient comfort, and improves image quality by reducing motion artifacts. Reduction of scan time is particularly important in cardiac imaging, for example, where it is highly desirable to acquire sufficient NMR data to reconstruct an image in a single breath hold.

Many different pulse sequences are known in the art for acquiring NMR signals from which an image may be reconstructed. Most of these pulse sequences sample k-space in a rectilinear pattern, but there is a class of pulse sequences which sample k-space in a spiral pattern. It is known that a spiral sampling pattern can be achieved by applying a sinusoidally varying readout magnetic field gradient during acquisition of each NMR signal and that spiral scanning methods can be used to rapidly acquire NMR data from which an image may be reconstructed. A spiral scanning method is also known wherein the sinusoidal readout gradient is shaped to more rapidly traverse the spiral sampling trajectory and, therefore, more rapidly sample k-space data. Scan time has been further reduced in the past by acquiring samples from little more than only one-half of k-space using interleaved spiral sampling trajectories. The missing k-space data are produced from a Hermitian approximation using the complex conjugate of the acquired k-space data or as described by D. C. Noll, et al., “Homodyne Detection in Magnetic Resonance Imaging” IEEE Transactions on Medical Imaging, vol. 10, No. 2, June 1991.

### SUMMARY OF THE INVENTION

NMR image data, from which an image can be reconstructed, are rapidly acquired in a magnetic resonance imaging (MRI) system in which a pulse sequence is performed to acquire NMR data that sample k-space in a trajectory comprised of a spiral segment that extends from the center of k-space to the periphery of k-space, and a symmetric spiral tail segment that extends from the center of k-space to sample only a central region of k-space. The pulse sequence may be repeated to sample along a plurality of interleaved trajectories such that the central region of k-space is substantially completely sampled and the periphery of k-space is only partially sampled. An image is produced from the resulting incomplete k-space data set using a homodyne reconstruction method.

### BRIEF DESCRIPTION OF THE DRAWINGS

FIG. 1 is a block diagram of an MRI system employing the invention;

FIG. 2 is a graphic representation of a preferred pulse sequence for practicing the invention; and

FIG. 3 is a graphic representation of the k-space sampling pattern performed by the pulse sequence of FIG. 2.

### DESCRIPTION OF THE PREFERRED EMBODIMENT

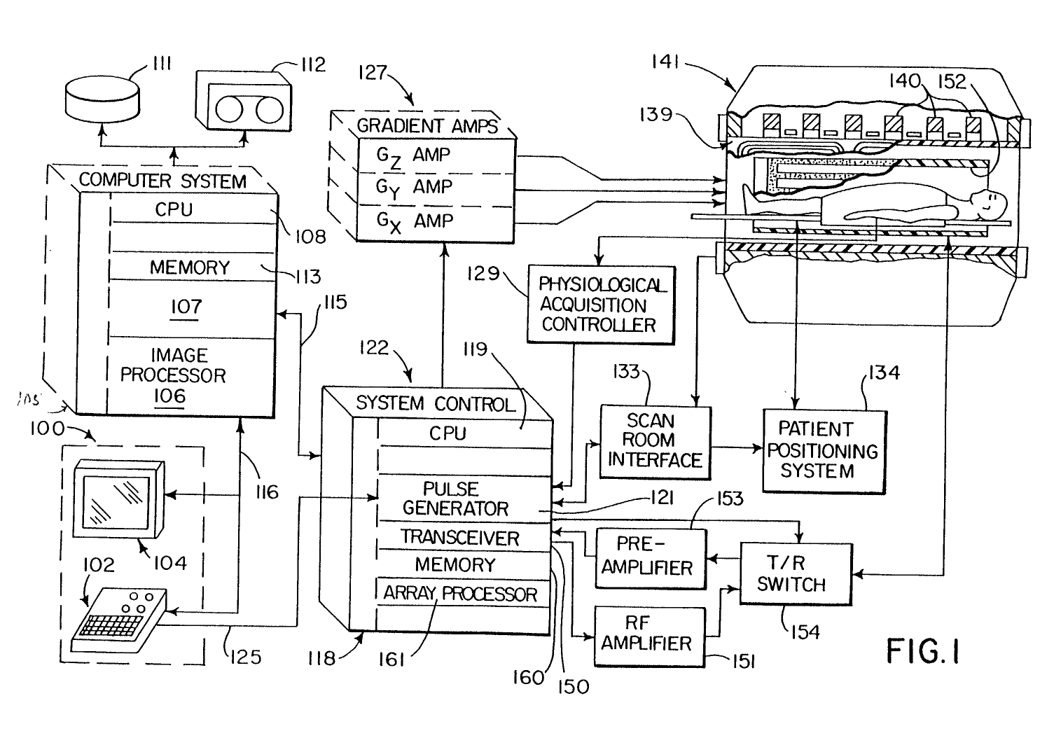

FIG. 1 illustrates the major components of an MRI system that incorporates the invention. Operation of the system is controlled from an operator console 100 which includes a keyboard and control panel 102 and a display 104. Console 100 communicates through a link 116 with a separate computer system 107 that enables an operator to control the production and display of images on a screen of display 104. Computer system 107 includes a number of modules which communicate with each other through a backplane 105. These include an image processor module 106, a CPU module 108, and a memory module 113 which is known in the art as a frame buffer for storing image data arrays. Computer system 107 is linked to a disk storage 111 and a tape drive 112 for storage of image data and programs, and communicates with a separate system control 122 through a high speed parallel link 115.

System control 122 includes a set of modules coupled together by a backplane 118. These include a CPU module 119 and a pulse generator module 121 which is coupled to operator console 100 through a serial link 125. System control 122 receives commands from the system operator through link 125 which indicate the scan sequence to be performed. Pulse generator module 121 operates the system components to carry out the desired scan sequence, producing data that indicate the timing, strength and shape of the RF pulses to be produced, and the timing of and length of the data acquisition window. Pulse generator module 121 is coupled to a set of gradient amplifiers 127 to control the timing and shape of the gradient pulses to be produced during the scan. Pulse generator module 121 also receives patient data from a physiological acquisition controller 129 that receives signals from sensors attached to the patient, such as ECG (electrocardiogram) signals from electrodes or respiratory signals from a bellows. Pulse generator module 121 is also coupled to a scan room interface circuit 133 which receives signals from various sensors associated with the condition of the patient and the magnet system. A patient positioning system 134 receives commands through the scan room interface circuit 133 to move the patient to the desired position for the scan.

The gradient waveforms produced by pulse generator module 121 are applied to gradient amplifier system 127 comprised of Gₓ, G_{y} and G_{z} amplifiers. Each gradient amplifier excites a corresponding gradient coil in a gradient coil assembly 139 to produce the magnetic field gradients used for position encoding acquired signals. Gradient coil assembly 139 forms part of a magnet assembly 141 which includes a polarizing magnet 140 and a whole-body RF (radio frequency) coil 152. Gradient amplifiers 127 are limited in amplitude of peak current they can provide and in the rate at which they can change current in gradient coils 139. As a result, the gradient field amplitude is limited, as is its slew rate.

A transceiver module 150 in system control 122 produces pulses which are amplified by an RF amplifier 151 and coupled to RF coil 152 by a transmit/receive switch 154. The resulting signals radiated by the excited nuclei in the patient may be sensed by the same RF coil 152 and coupled through transmit/receive switch 154 to a preamplifier 153. The amplified NMR signals are demodulated, filtered, and digitized in the receiver section of transceiver 150. Transmit/receive switch 154 is controlled by a signal from pulse generator module 121 to electrically couple RF amplifier 151 to coil 152 for the transmit mode and to preamplifier 153 for the receive mode. Transmit/receive switch 154 also enables a separate RF coil (for example, a head coil or surface coil, not shown) to be used in either the transmit or receive mode.

The NMR signals picked up by RF coil 152 are digitized by transceiver module 150 and transferred to a memory module 160 in system control 122. The receiver in transceiver module 150 preserves the phase of the acquired NMR signals in addition to signal magnitude. The down converted NMR signal is applied to an analog-to-digital (A/D) converter (not shown) which samples and digitizes the analog NMR signal. The samples are applied to a digital detector and signal processor (not shown) which produces 16-bit in-phase (I) values and 16-bit quadrature (Q) values corresponding to the received NMR signal. The resulting stream of digitized I and Q values of the received NMR signal are supplied through backplane 118 to memory module 160 where they are employed to reconstruct an image. For a more detailed description of the receiver, reference is made to Stormont et al. U.S. Pat. No. 4,992,736, issued Feb. 12, 1991, assigned to the instant assignee, and which is incorporated herein by reference.

When the scan is completed and an entire array of data has been acquired in memory module 160, an array processor 161 operates to grid the data into an array when necessary, and Fourier transform the data into an array of image data which is conveyed through link 115 to computer system 107 where the data are stored in disk memory 111. In response to commands received from operator console 100, these image data may be archived on tape drive 112, or may be further processed by image processor 106 and conveyed to operator console 100 for presentation on display 104.

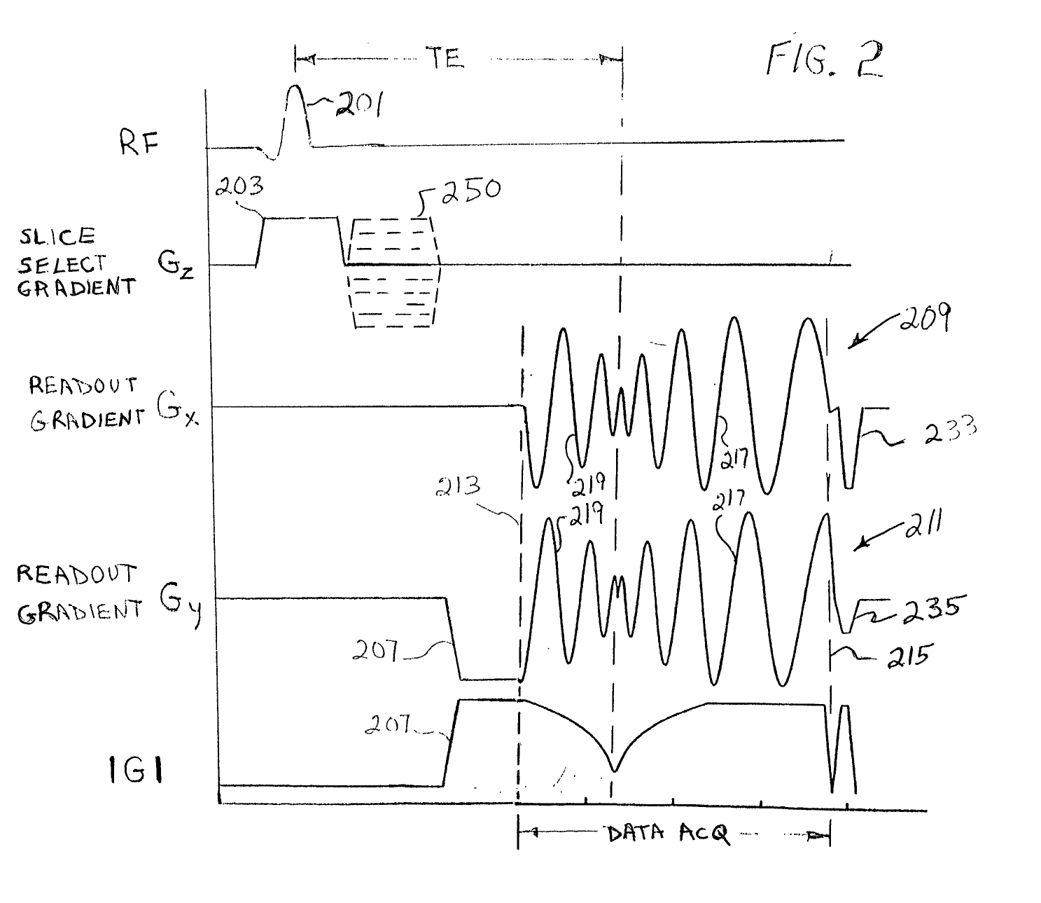

The MRI system of FIG. 1 is employed to acquire NMR data using the pulse sequence of FIG. 2. As explained above, this pulse sequence is performed under the direction of pulse generator module 121 which directs the system components to produce the indicated RF pulses and gradient waveforms.

As shown in FIG. 2, the preferred pulse sequence is a two-dimensional, gradient recalled echo pulse sequence including a selective RF excitation pulse 201 that is produced in the presence of a G_{z} slice select gradient pulse 203 to produce transverse magnetization in a selected slice of spins in the subject to be imaged.

A readout gradient dephasing pulse 207 is produced following the transverse excitation and is immediately followed by sinusoidal readout gradient waveforms 209 and 211 that produce time varying magnetic field gradients along the respective Gₓ and G_{y} gradient axes. An NMR echo signal peak is produced at an echo time TE, and the NMR echo signal is acquired during the interval indicated by dashed lines 213 and 215.

Each of the time varying readout gradient waveforms 209 and 211 is comprised of two waveform segments: a spiral waveform segment 217 that is played out during the data acquisition window after echo time TE; and a symmetric spiral tail waveform segment 219 that is played out during the data acquisition window prior to the echo time TE. The spiral waveform segment 217 is derived from the k-space Archemedian spiral of the form:

kₓ(t)=a(t) cos [a(t)]

k_{y}(t)=a(t) sin [a(t)] (1)

The readout gradient waveform amplitude is related to the velocity at which the k-space spiral sampling trajectory is sampled by the following equations:

Gₓ(t)=(dkₓ(t)/dt)/γ

G_{y}(t)=(dk_{y}(t)/dt)/γ. (2)

The readout gradient waveform slew rate is related to the acceleration at which the k-space spiral sampling trajectory is sampled by the following equations:

dGₓ(t)/dt=(d²kₓ(t)/dt²)/γ

dG_{y}(t)/dt=(d²k_{y}(t)dt²)/γ (3)

Given the amplitude and slew rate limitations of the gradient system hardware, the function a(t) in equation (1) is determined numerically by solving the differential equation that relates the maximum gradient slew rate and maximum gradient amplitude to the velocity and acceleration of k-space sampling.

One technique for estimating a low frequency field map is to use two different TE times. Another technique samples the symmetric spiral tail waveform 219, which employs the same function a(t) and samples a spiral trajectory which is the k-space complement of the sampling trajectory of spiral waveform segments 217. This symmetric spiral tail segment has the form:

kₓ(t)=a(t−t′) cos [a(t−t′)]

k_{y}(t)=a(t−t′) sin [a(t−t′)], (4)

where t′ is a constant that determines the size of central k-space that is sampled by the symmetric spiral tail segment 219.

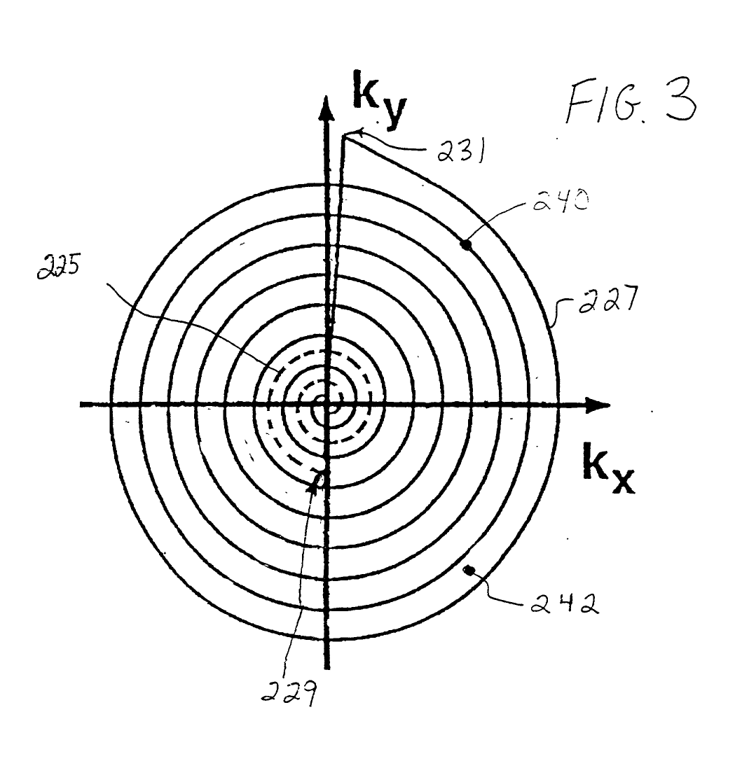

The k-space sampling trajectory performed by the pulse sequence of FIG. 2 is shown in FIG. 3 to include a symmetric spiral tail segment indicated by dashed line 225 and a spiral segment indicated by solid line 227. As the time varying readout gradients 209 and 211 (FIG. 2) are played out during the data acquisition period, sampling begins at a k-space location 229 and spirals inward along a trajectory 225, reaching the center of k-space at the echo time TE. Spiral waveform segment 217 is then played out and sampling spirals outward along trajectory 227 toward the periphery of k-space. The sampling is completed at the periphery of k-space at location 231. Rephasing readout gradient pulses 233 and 235 (FIG. 2) are applied after the data acquisition window to prepare the transverse magnetization for the next repetition of the pulse sequence. RF spoiling is employed to null transverse magnetization prior to execution of the next pulse sequence.

While it is possible to acquire an image in a single spiral pulse sequence, it is much more common to perform a plurality of spiral pulse sequences in which the spiral sampling trajectories are interleaved to uniformly sample k-space. If fewer spiral trajectories or “arms” are sampled, then each spiral trajectory must encircle, or “wrap” around the center of k-space more times to adequately sample k-space. Table A lists a number of spiral interleave combinations which produce good reconstructed images.

TABLE A

| | | | | Total | Tail |
|---|---|---|---|---|---|
| Spiral | | Total | Tail | Readout | Readout |
| Arms | Wraps | Samples | Samples | Time (μs) | Time (μs) |
| 21 | 3 | 316 | 30 | 1.264 | .12 |
| 15 | 4 | 463 | 38 | 1.852 | .152 |
| 11 | 5 | 610 | 46 | 2.44 | .184 |
| 9 | 6 | 757 | 52 | 3.028 | .208 |
| 7 | 7 | 905 | 60 | 3.62 | .24 |
| 7 | 8 | 1053 | 68 | 4.212 | .27 |
| 5 | 9 | 1348 | 82 | 5.392 | .328 |

From FIG. 3, it should be apparent that because the symmetric tail segment 225 only samples the central region of k-space, this central region is sampled with twice the density as the surrounding k-space peripheral region. In a preferred embodiment the central region is sampled to provide the desired image resolution and SNR (signal-to-noise ratio) and the peripheral region is thus under-sampled. The missing peripheral k-space samples are produced by calculating the complex conjugate of the k-space data acquired by the outer portion of the spiral segments 227. For example, if a signal sample

S=I+Q

is acquired at a point 240 on the spiral trajectory 227, its complex conjugate signal

S*=I−Q

fills in for the missing peripheral k-space data at point 242. A complete k-space data set is thus formed and used to reconstruct an image using the homodyne method described in the above-cited Noll et al publication. It has been found that the increased sampling of the center of k-space provided by the symmetric tail segments can be used to eliminate phase errors that are introduced at low spatial frequencies near the origin of k-space. MRI systems typically have slow variations in the magnetic fields which they produce, and these variations may cause image artifacts when complex symmetry near the origin of k-space is used in image reconstruction. These variations are confined to low spatial frequencies and the symmetric tail segments need not extend far from the center of k-space.

Use of the present invention reduces the spiral scan time by nearly 50%. A 50% reduction in scan time could be achieved by simply sampling one-half of k-space using spiral sampling trajectory 227 alone. However, unacceptable image artifacts may be produced. By adding the symmetric spiral tail trajectory 225 to the pulse sequence, such image artifacts are eliminated or substantially reduced with an increase in scan time of less than 10%, as shown in Table A.

Many variations are possible from the preferred embodiments described above. For example, a three-dimensional image may be acquired by adding phase encoding in the slice selected direction as indicated in FIG. 2 by dashed lines 250. For each separate G_{z} phase encoding value kₓ, k_{y} space is sampled using one or more spiral trajectories as described above. The process is repeated for each G_{z} phase encoding value (e.g. 16 values) until a 3D k-space data set is acquired.

The invention may also be used with other spin echo pulse sequences. Other RF excitation methods for producing transverse magnetization may also be used. Such methods include spectral-spatial excitation. Also, one or more gradient axis may be flow compensated by the addition of gradient moment nulling pulses as described in Glover et al. U.S. Pat. No. 4,731,583, issued Mar. 15, 1988 and assigned to the instant assignee.

While only certain preferred features of the invention have been illustrated and described, many modifications and changes will occur to those skilled in the art. It is, therefore, to be understood that the appended claims are intended to cover all such modifications and changes as fall within the true spirit of the invention.

1. A method for producing an image with a magnetic resonance imaging system, comprising:
a) establishing a polarizing magnetic field in a subject to be imaged;
b) generating an RF excitation pulse to produce transverse magnetization in the subject;
c) applying magnetic field gradients to the subject during an acquisition period such that k-space is sampled in a trajectory comprised of a spiral segment that extends from the center of k-space to the periphery of k-space, and a symmetric, spiral tail segment that extends from the center of k-space to sample only a central region of k-space;
d) acquiring an NMR signal during the acquisition period; and
e) reconstructing an image from the acquired NMR signal.

2. The method as recited in claim 1 wherein steps a), b), c) and d) are repeated and step e) is performed using all the acquired NMR signals.

3. The method as recited in claim 2 wherein the step of applying magnetic field gradients to the subject is performed so that k-space is sampled in a plurality of interleaved trajectories.

4. The method as recited in claim 3 wherein the step of applying magnetic field gradients to the subject is performed so that the central region of k-space is fully sampled and the surrounding peripheral region of k-space is partially sampled.

5. The method as recited in claim 4 wherein the step of reconstructing an image comprises:
filling in the sampled region of k-space with the complex conjugate of acquired NMR signal samples to form a complete k-space data set; and
Fourier transforming the complete k-space data set.

6. The method as recited in claim 4 wherein the periphery of k-space is partially sampled by:
sampling k adjacent spiral trajectories, wherein a complete data set comprises n>k symmetrically placed spiral trajectories, each rotated by 2π/n radians from an adjacent spiral trajectory; and
sampling alternate spiral trajectories.

7. The method as recited in claim 1 wherein the step of applying magnetic field gradients to the subject is performed by first sampling the symmetric spiral tail segment and then sampling the spiral segment.

8. The method as recited in claim 7 wherein the step of applying magnetic field gradients to the subject is performed such that the symmetric spiral tail segment is sampled along a path toward the center of k-space and the spiral segment is sampled along a path away from the center of k-space.

9. The method as recited in claim 1 including the step of generating a low frequency field map using two low frequency images obtained with different TE echo times.

10. A magnetic resonance imaging system, comprising:
a magnet system for producing a polarizing magnetic field in a subject;
means for producing an RF excitation pulse to establish transverse magnetization in the subject;
a magnetic field gradient assembly for applying to the subject during an acquisition period time varying magnetic field gradients to sample k-space in a trajectory comprised of a spiral segment that extends from the center of k-space to the periphery of k-space, and a symmetric, spiral tail segment that extends from the center of k-space to sample only a central region of k-space;
a receiver for acquiring an NMR signal during the acquisition period; and
means for reconstructing an image from the acquired NMR signal.

11. The magnetic resonance imaging system of claim 10 wherein the magnetic field gradient assembly is adapted to apply the time varying magnetic field gradients for producing the symmetric spiral tail segment for less than ten percent of said acquisition period.

12. The magnetic resonance imaging system as recited in claim 10 including a pulse generator for controlling the means for producing an RF excitation pulse, the magnetic field gradient assembly and the receiver, and the means for reconstructing an image from the acquired NMR signal is adapted to reconstruct said image after a single pulse sequence.

13. The magnetic resonance imaging system as recited in claim 12 wherein the pulse generator is operable to repeat the pulse sequence a plurality of times to acquire a plurality of NMR signals, and the means for reconstructing an image from the acquired NMR signal is adapted to employ all of the acquired NMR signals.

14. The magnetic resonance imaging system as recited in claim 10 wherein said means for producing an RF excitation pulse comprises a transceiver, said receiver comprises a portion of said transceiver, and said means for reconstructing an image comprises a display and an image processor coupled to said display.
